(19) United States
(12) Patent Application Publication
(10) Pub. No.: US 2022/0087670 A1
(43) Pub. Date: Mar. 24, 2022

(54) PROSTHETIC IMPLANT

(71) Applicant: BIORL S.R.L., Melzo (Milano) (IT)

(72) Inventor: Redouane SELMOUNE, Bollate (Milano) (IT)

(21) Appl. No.: 17/357,285

(22) Filed: Jun. 24, 2021

(30) Foreign Application Priority Data
Sep. 24, 2020 (IT) 102020000022540

(51) Int. Cl. A61B 17/04; A61F 2/28

(52) U.S. Cl. CPC A61B 17/0401; A61F 2/28; A61B 2017/0414; A61F 2002/30461; A61B 2017/0459; A61B 2017/0464; A61B 2017/0456

(57) ABSTRACT

Prosthetic implant including a solid portion shaped like a first portion of a human skeletal structure and a suture portion, connected to the solid portion, and shaped like a second portion of a human skeletal structure complementary to the first portion. The suture portion includes a suture body, having a multitude of interconnected internal cavities within which a suture instrument is directed, or can be directed, through an external surface of the suture body. The suture body further includes a multitude of holes uniformly distributed on the external surface of the suture body and opening into the internal cavities. The external surface of the suture body defines a suture surface of the prosthetic implant configured to enable biological tissue to be sewn to the prosthetic implant.

This application claims priority to Italian Patent Application 102020000022540 filed Sep. 24, 2020, the entirety of which is incorporated by reference herein.

The present invention relates to a prosthetic implant which is widely used in the medical field, particularly in the field of medical devices for large segments.

As known, during an individual's life, the human body may be subjected to various traumas, inflammations and diseases that damage and deteriorate certain bones, resulting in very serious disorders and inflammations.

It is therefore possible for a person to develop damage over time at a joint (e.g., hip, shoulder, etc.) resulting in broken or deteriorated bone, irritated bursae or worn cartilage. In particular, damaged cartilage can lead to various forms of arthritis, such as osteoarthritis, rheumatoid arthritis and traumatic arthritis.

Nowadays, these diseases can be healed by physical treatment (exercise, physical therapy, external support) or medical treatment (through the administration of specific drugs). However, such treatments are sometimes not sufficient and, therefore, a complete replacement of the injured part with a medical prosthesis is taken into consideration.

As known, there exist different medical prostheses depending on the severity of the patient's pathology. In particular, first intention prostheses are known to be used where there is worn cartilage but no major bone wear or deformities. “Revision” prosthetic implants are also known to be used when the patient has a morphology that does not meet the requirements for a first intention implant. Finally, medical implants for large segments are known which tend to be used to treat cancer patients or patients who have suffered from severe infections resulting in significant bone resorption and a subsequent loss of large bone segments.

As known, prosthetic implants are made of different materials, e.g., hip prostheses have a titanium alloy stem and acetabular cup, a ceramic head and a polyethylene insert.

Referring now in particular to medical implants for large bone or tumour segments, to which the prosthetic implant of the present invention pertains, they typically have components comprising a lining having a number of holes, typically three, which allow to attach the body tissues surrounding the prosthesis to the prosthesis itself. These holes are distributed on the prosthesis and are divided between each other so that one hole is interconnected with a limited number of other holes placed in close proximity to allow entering and exiting the suture thread.

Disadvantageously, known prosthetic implants made as such are difficult to adapt to the different anatomies of patients as, when the prosthesis is fixed in the patient's cavity, the tissues surrounding it are joined to the prosthesis by sutures applied through the holes present on the prosthesis.

As each hole is interconnected with a limited number of other holes on the prosthesis, the doctor does not have complete freedom in placing sutures. In case, for instance, due to a particular position of the prosthesis consequent to a specific anatomy of a patient, the doctor needs to insert the suture instrument into a certain hole and to extract such instrument out at a distal position, he would be prevented from doing so because holes may not have been made in that position or, if present, may not be communicating with the hole through which the suture instrument was inserted.

Thus, in these circumstances, sutures are made in positions and/or of a length that are not satisfactory if compared to what required.

The structure rigidity of known implants does not thus allow the surgeon to be totally free in the positioning and in the number of sutures to be applied, to the detriment of an optimal joint stability.

A further disadvantage is that known prostheses do not optimally promote tissue vascularisation. It is well known that once the prosthetic implant has been installed in the patient, a process of osseointegration is triggered which results in the implant being incorporated into the patient's skeletal structure. During this process, the patient's bone “grows again” within the holes of the aforesaid lining. The known linings, however, as already mentioned, have a very limited number of holes and, therefore, have a poorly “porous” structure, i.e., a structure that is not adapted to ensure sufficient blood supply to the tissues to promote a proper bone regrowth.

The technical task of the present invention is therefore to make available a prosthetic implant capable of overcoming the drawbacks resulting from the prior art.

The object of the present invention is, therefore, to make available a prosthetic implant that allows to obtain an optimal anatomical positioning and fixation while taking into account the physiological characteristics of the patient.

A further object of the present invention is to make available a prosthetic implant which allows the surgeon to have greater freedom with regard to the number of sutures applicable and their positioning.

In addition, a further object of the present invention is to make available a prosthetic implant that promotes a better vascularisation of tissues.

A further object of the present invention is to make available a prosthetic implant that ensures a better adhesion of the tissues to the prosthetic implant.

The technical task specified and the objects specified are substantially achieved by a prosthetic implant comprising the technical features set forth in one or more of the appended claims. The dependent claims correspond to possible embodiments of the invention.

Further characteristics and advantages of the present invention will become clearer from the indicative and therefore non-limiting description of an embodiment of a prosthetic implant.

Such description will be set forth herein below with reference to the accompanying drawings, provided for merely indicative and non-limiting purposes, wherein:

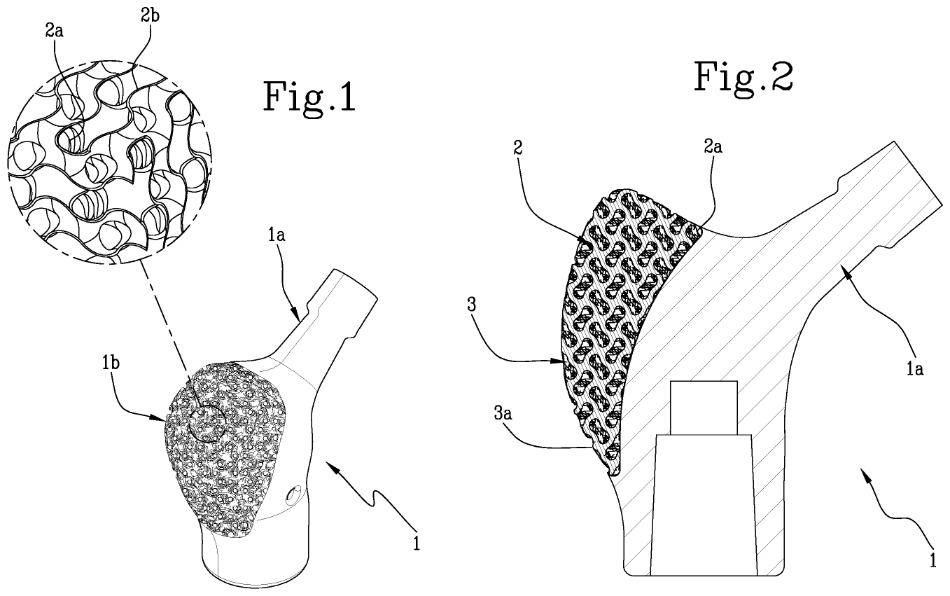

FIG. 1 shows a perspective view of a prosthetic implant; and

FIG. 2 shows a section view of the prosthetic implant of FIG. 1.

With reference to the attached figures, 1 indicates a prosthetic implant.

The prosthetic implant object of the present invention comprises a solid portion 1a shaped like a first portion of a human skeletal structure and a suture portion 1b connected to the solid portion 1a and shaped like a second portion of the human skeletal structure that is complementary to the first portion.

In other words, the first portion and the second portion are form-coupled.

In the embodiment shown in the enclosed figures, preferably the solid portion 1a and the suture portion 1b are shaped to define the geometry of a proximal femur.

In particular, the solid portion 1a has a typical shape of the diaphyseal zone and the calcar zone up to the femoral neck. The suture portion 1b has a substantially trabecular structure and is shaped like the trochanteric area of the proximal femur. Preferably, as illustrated in the enclosed figures, the suture portion 1b extends both at the front and at the back of the implant 1 as well as at a proximal lateral region of the implant 1 itself.

In further embodiments, however, which are not illustrated, the solid portion 1a and the suture portion 1b may be respectively shaped like a first and a second portion of any skeletal structure such as, for example, the structure of an ankle, a knee, a shoulder and the like.

The suture portion 1b is positioned on the implant 1 so as to occupy a position suitable to be arranged in use at the biological tissues to be sutured, i.e., a position where the prosthetic implant 1 is to be fixed to the soft tissues or skeletal structure of a patient by means of sutures. This positioning varies depending on the portion of the patient's skeleton that the prosthetic implant 1 is intended to replace. This positioning may also depend on the size of the prosthesis and/or may be ‘tailor-made’ for a particular patient. For example, in the embodiment illustrated in the enclosed figures, the suture portion 1b is shaped like the greater trochanter of the femur as this is the portion that is normally used as the connection point between the prosthetic implant 1 and the remaining skeletal structure and soft tissues of the patient.

The suture portion 1b comprises a suture body 2 having a multitude of interconnected internal cavities 2a within which a suture instrument, such as a surgical needle, is directed or can be directed through an external surface 3 of the suture body 2 itself.

In other words, the internal cavities 2a have such a shape as to allow the suture instrument to pass from one internal cavity 2a to another.

As shown in the enlargement of FIG. 1, the internal cavities 2a present in the suture body 2 branch out within the entire volume occupied by the suture body 2 itself, forming a substantially porous structure in which each internal cavity 2a is interconnected with all the other internal cavities 2a to allow the surgeon to direct the suture instrument more freely and flexibly.

Preferably, a ratio between the volume of the suture body 2 and the volume occupied by the internal cavities 2a ranges between 45% and 65%, preferably approximately 55%.

The ratio between the above volumes intends to define a porosity measure of the suture portion 1b to determine what percentage of the entire volume of the suture body 2 is occupied by the internal cavities 2a.

Advantageously, the structure of the suture portion 1b, and, therefore, the structure of the suture body 2, being highly porous, is lighter than the known structures, advantageously lightening the prosthetic implant 1 (making it, as such, lighter than a prosthetic implant of the known type in which the suture portion is solid).

Preferably, moreover, the suture portion 1b extends within a smaller volume than the volume of the solid portion 1a. In other words, the solid portion 1a occupies more volume than the suture portion 1b.

In a preferred embodiment, the suture body 2 is shaped like a gyroid so as to generate an interconnected porous geometry.

In a preferred embodiment, the internal cavities 2a have a basically circular cross-section having a diameter ranging between 1 mm and 4 mm, preferably approximately 1.5 mm.

These internal cavities 2a are also bounded by walls 2b having a thickness ranging between 0.3 mm and 1.5 mm.

In other words, the walls 2b define the “solid part” of the suture body 2, while the internal cavities 2a define the “empty part” of the suture body 2.

Advantageously, the internal cavities 2a are sized to promote adequate vascularisation of the patient's organic tissues and to allow rapid bone regeneration, i.e., rapid osseointegration of the prosthetic implant 1.

As shown in the enclosed figures, the suture body 2 further comprises a plurality of holes 3a made in an evenly distributed manner on the external surface 3 of the suture body itself 2 and opening into the internal cavities 2a.

The external surface 3 of the suture body 2 defines, in fact, a suture surface of the prosthetic implant 1 configured to enable biological tissue to be sewn to said prosthetic implant 1.

In other words, the holes 3a are distributed on the external surface 3 in such a way as to occupy the entire extent of the external surface 3 serving as access and exit points from the internal cavities 2a for the suturing instrument during the process of suturing the biological tissues onto the prosthetic implant 1.

In a preferred embodiment, such holes 3a may be distributed on the external surface 3 according to a regular matrix in which each hole 3a is spaced from the others of a predetermined amount.

When suturing the organic tissues on the implant 1, the surgeon chooses the hole 3a where to insert the suture instrument. Once the suture instrument has been inserted, the surgeon directs it into the internal cavities 2a of the suture body 2. Since the internal cavities 2a are mutually interconnected, the surgeon can extract the suture instrument out of any hole 3a occupying a desired position on the external surface 3.

In other words, the surgeon can insert the suture instrument into any hole 3a in the external surface 3 and extract it out of any other hole 3a by passing through any number of internal cavities 2a.

Thanks to the structure of the suture body 2, it is therefore possible to insert the suture instrument within a given hole 3a and to move this instrument within the internal cavities 2a so as to define any path in order to extract the suture instrument from a hole 3a occupying the desired position on the external surface 3.

Advantageously, this allows for greater surgical flexibility and improved suture efficiency, being the suture made exactly where it is needed.

Advantageously, the shape of the internal cavities 2a of the suture body 2 and the distribution of the holes 3a on the external surface 3 allow the surgeon to suture the patient's organic tissues along the entire extent of the external surface 3 of the suture portion 1b of the prosthetic implant 1, as it is possible to move the suture instrument into and out of substantially any point on the external surface 3 itself.

In such a situation, therefore, the surgeon can apply a large number of sutures, placing them where most convenient along the entire suture surface 3. This results in a better adhesion of the organic tissues to the implant 1 and in a better stability of the prosthetic implant 1 itself.

According to a further aspect of the present invention, the solid portion 1a is made by an additive manufacturing technique, preferably by Electron Beam Melting (EBM) or Direct Metal Laser Melting (DMLS).

The suture portion 1b can also be made by an additive manufacturing technique, preferably by Electron Beam Melting (EBM) or Direct Metal Laser Melting (DMLS).

Advantageously, additive manufacturing techniques make it easier, faster and more accurate to create the internal cavities 2a of the suture body 2 if compared to the known techniques.

In particular, by means of additive manufacturing techniques it is possible to reproduce in the suture body 2 a bone structure that is very similar to the real one, thus promoting bone regrowth once the prosthetic implant 1 has been installed in the patient.

Preferably, the suture portion 1b is made in a single piece with the solid portion 1a, still more preferably by a single additive manufacturing process.

Such choice of construction makes it possible to create a particularly strong and stable prosthetic implant.

In a further embodiment, the suture portion 1b and the solid portion 1a are made separately and subsequently assembled.

According to a further possible embodiment, the suture portion 1b may be applied to retrofit an existing prosthetic implant 1 from which the portion corresponding to the suture portion 1b has been removed.

According to these further aspects, it is possible to upgrade a pre-existing prosthetic implant in such a way as to improve its functioning, or to produce the individual components which can also be supplied as a kit or otherwise assembled as required.

Preferably, the solid portion 1a and the suture portion 1b are made of a titanium alloy, in particular of an alloy comprising Titanium, Aluminium and Vanadium which can be made according to the formula Ti₆Al₄V.

The present invention achieves the intended objects overcoming the drawbacks of the known art.

In particular, the prosthetic implant 1 allows to carry out tissue suturing along the entire extent of the external surface 3 of the suture portion 1b enabling the surgeon to freely choose the positioning and the size of the sutures.

A further advantage derives from the possibility of making the suture portion 1b by means of additive manufacturing techniques, as it enables to obtain quickly and in an extremely precise way, a substantially porous structure which makes it possible to insert into and extract the suture instrument out of any point on the external surface 3 of the suture body 2.

Advantageously, the possibility of making the suture portion 1b using additive manufacturing techniques allows accurate control over its size, so as to obtain a structure which promotes the osseointegration of implant 1 and the tissue vascularisation.

A further advantage is that the suture portion 1b allows a better adhesion of the tissues to the implant 1 itself, as well as a better stability of the entire implant 1 within the patient's body.

1. A prosthetic implant comprising:
a solid portion shaped like a first portion of a human skeletal structure, and
a suture portion connected to the solid portion and shaped like a second portion of a human skeletal structure that is complementary to the first portion;
wherein said suture portion comprises a suture body having:
a multitude of interconnected internal cavities within which a suture instrument is directed, or can be directed, through an external surface of said suture body, and
a multitude of holes uniformly distributed on the external surface of said suture body and opening into said internal cavities,
said external surface of the suture body defining a suture surface of the prosthetic implant configured to enable biological tissue to be sewn to said prosthetic implant.

2. The implant according to claim 1, wherein the suture portion is made using an additive manufacturing technique, preferably using Electron Beam Melting (EBM) or Direct Metal Laser Melting (DMLS) procedures.

3. The implant according to claim 1, wherein the solid portion is made using an additive manufacturing technique, preferably using Electron Beam Melting (EBM) or Direct Metal Laser Melting (DMLS) procedures.

4. The implant according to claim 1, wherein said at least one suture portion is made of a single piece with said solid portion, preferably using a single additive manufacturing process.

5. The implant according to claim 1, wherein said solid portion and said suture portion are made of titanium alloy, in particular an alloy comprising titanium, aluminium, and vanadium according to the Ti₆Al₄V formula.

6. The implant according to claim 1, wherein each cavity has a basically circular cross-section with a diameter ranging between 1 mm and 4 mm, preferably approximately 1.5 mm.

7. The implant according to claim 1, wherein said cavities are bounded by walls of the suture body that are between 0.3 mm and 1.5 mm thick.

8. The implant according to claim 1, wherein said suture portion extends within a smaller volume than the volume of the solid portion.

9. The implant according to claim 1, wherein a ratio between the volume of said suture body and the volume occupied by said internal cavities ranges between 45% and 65%, preferably about 55%.
